(19) United States
(12) Patent Application Publication
(10) Pub. No.: US 2023/0165527 A1
(43) Pub. Date: Jun. 1, 2023

(54) CBT-I TREATMENT SYSTEM AND OPERATION METHOD THEREOF

(71) Applicant: HONEYNAPS CO., LTD., Seoul (KR)

(72) Inventors: Young Jun LEE, Seoul (KR); Tae Kyoung HA, Seoul (KR)

(73) Assignee: HONEYNAPS CO., LTD., Seoul (KR)

(21) Appl. No.: 18/071,390

(22) Filed: Nov. 29, 2022

(30) Foreign Application Priority Data
Nov. 30, 2021 (KR) 10-2021-0168585

(51) Int. Cl. A61B 5/00; A61B 5/024

(52) U.S. Cl. CPC A61B 5/6802; A61B 5/02416

(57) ABSTRACT

Disclosed are a CBT-I treatment system for the treatment of insomnia and an operation method thereof. In accordance with an aspect of the present disclosure, a CBT-I treatment server including a database configured to store a plurality of CBT-I programs for insomnia treatment; a sleep evaluator configured to generate sleep evaluation data of an insomnia patient based on biosignal data including a change in a biosignal during sleep of the insomnia patient; a CBT-I prescriber configured to determine a CBT-I program for insomnia treatment of the insomnia patient from among the plural CBT-I programs based on the sleep evaluation data and to generate a CBT-I prescription including the determined CBT-I program; and a communicator configured to receive a biosignal from a biosignal collection device for generating biosignal data by sensing a change in a biosignal during sleep of the insomnia patient and to transmit the CBT-I prescription to an insomnia patient's terminal is provided.

### CROSS-REFERENCE TO RELATED APPLICATION

This application claims priority to Korean Patent Application No. 10-2021-0168585, filed on Nov. 30, 2021 in the Korean Intellectual Property Office, the disclosure of which is incorporated herein by reference.

### BACKGROUND OF THE DISCLOSURE

### Field of the Disclosure

The present disclosure relates to a CBT-I treatment system for the treatment of insomnia and an operation method thereof.

### Description of the Related Art

Currently, the treatment of insomnia is largely divided into drug treatment and non-drug treatment. Drug treatment for insomnia is the most common treatment method at home and abroad. Drug treatment for insomnia works as a sleep-inducing mechanism by changing neurotransmitters that affect sleep and wakefulness. Examples of neurotransmitters for inducing wakefulness include norepinephrine, serotonin, acetylcholine, histamine, and hypocretin/orexin, and examples of neurotransmitters for inducing sleep include maenosine, GABA, galanin, and melatonin. Drugs for treating insomnia often include an antagonist for lowering wakefulness or a benzodiazepine receptor agonist for inducing sleep. However, due to the side effects of drug treatment, long-term use of more than 5 weeks is not recommended.

Cognitive-Behavior Therapy (hereinafter referred to as “CBT-I”) for insomnia is a non-pharmacological treatment that is recognized by the United States Department of Health and Human Services as the most effective treatment method and is recommended as a primary treatment. CBT-I aims to treat insomnia by eliminating the habit, cognitive and environmental factors that cause and persist insomnia. CBT-I is a short-term treatment which consists of 4 to 8 sessions on average, and is divided into items such as sleep education, sleep hygiene, stimulus control, sleep restriction, relaxation therapy, cognitive therapy, and light therapy. Many therapists show improvement in insomnia by simply performing these treatment items mechanically, but implementing these items as a formula may not be effective in terms of time and money.

Although CBT-I shows good treatment results for insomnia, patients have to visit the hospital for 4 to 8 weeks to receive continuous treatment for the actual treatment of insomnia and the issue of compliance with lighting treatment, which requires daily recording of patient's sleep habits in a sleep log during the treatment process, is raised. In addition, there is a problem to be improved in the lighting treatment that does not reflect the individual characteristics of patients due to the uniformity of the existing CBT-I program.

### SUMMARY OF THE DISCLOSURE

Therefore, the present disclosure has been made in view of the above problems, and it is an object of the present disclosure to provide a technique for treating insomnia that reflects the individual characteristics of insomnia patients.

It is another object of the present disclosure to provide a technology capable of treating insomnia without requiring an insomnia patient to visit a hospital or face a specialist such as a doctor.

In accordance with an aspect of the present disclosure, the above and other objects can be accomplished by the provision of a CBT-I treatment server, including: a database configured to store a plurality of CBT-I programs for insomnia treatment; a sleep evaluator configured to generate sleep evaluation data of an insomnia patient based on biosignal data including a change in a biosignal during sleep of the insomnia patient; a CBT-I prescriber configured to determine a CBT-I program for insomnia treatment of the insomnia patient from among the plural CBT-I programs based on the sleep evaluation data and to generate a CBT-I prescription including the determined CBT-I program; and a communicator configured to receive a biosignal from a biosignal collection device for generating biosignal data by sensing a change in a biosignal during sleep of the insomnia patient and to transmit the CBT-I prescription to an insomnia patient's terminal.

In one embodiment, the biosignal data may include at least one of electoencephalogram (EEG), electrocardiogram (ECG), electromyogram (EMG), electrooculogram (EOG) and photoplethysmogram (PPG).

In one embodiment, the CBT-I treatment server may further include a sleep diary manager configured to generate a sleep diary of the insomnia patient based on the sleep evaluation data, wherein the communicator transmits the sleep diary to the insomnia patient's terminal.

In one embodiment, the communicator may further receive at least one of age information, gender information, smoking information, drinking level, exercise information, caffeine information, and nap information of the insomnia patient from the insomnia patient's terminal, and the CBT-I prescriber may further include at least one of age information, gender information, smoking information, drinking level, exercise information, caffeine information, and nap information of the insomnia patient and may determine a CBT-I program for insomnia treatment of the patient.

In one embodiment, the communicator may transmit the CBT-I prescription to an expert terminal and may receive feedback, inputted by an insomnia treatment expert, for the CBT-I prescription from the expert terminal.

In one embodiment, the CBT-I prescriber, when the feedback includes correction information on the CBT-I prescription, may correct the determined CBT-I program based on the correction information, and may generate a corrected CBT-I prescription including the corrected CBT-I program, and the communicator may transmit the corrected CBT-I prescription to the insomnia patient's terminal.

In one embodiment, when the feedback does not include correction information on the CBT-I prescription, the communicator may transmit the CBT-I prescription including the determined CBT-I program to the insomnia patient's terminal.

In accordance with another aspect of the present disclosure, there is provided a method of operating a CBT-I treatment server including a database for storing a plurality of CBT-I programs for insomnia treatment, the method including: receiving a biosignal from a biosignal collection device for collecting biosignal data including a change in a biosignal during sleep of an insomnia patient; generating sleep evaluation data of the insomnia patient based on the biosignal data including the change in the biosignal during sleep of the insomnia patient; determining a CBT-I program for insomnia treatment of the insomnia patient from among the plural CBT-I programs based on the sleep evaluation data and generating a CBT-I prescription including the determined CBT-I program; and transmitting the CBT-I prescription to an insomnia patient's terminal.

In accordance with yet another aspect of the present disclosure, there is provided a CBT-I treatment system, including: a biosignal collector configured to collect biosignal data including a change in a biosignal during sleep of an insomnia patient and to transmit the collected biosignal data; a CBT-I treatment server configured to receive the biosignal data from the biosignal collector to generate sleep evaluation data of the insomnia patient based on the biosignal data, to determine a CBT-I program for insomnia treatment of the patient from among the plural CBT-I programs based on the sleep evaluation data, and to generate a CBT-I prescription including the determined CBT-I program; and an insomnia patient's terminal configured to receive the CBT-I prescription from the CBT-I treatment server and to output the CBT-I program included in the CBT-I prescription.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1 is a view for explaining a CBT-I treatment system according to an embodiment of the present disclosure;

FIG. 2 illustrates a block diagram of a CBT-I treatment server according to an embodiment of the present disclosure;

FIG. 3 is a view for explaining an operation of a CBT-I treatment system according to an embodiment of the present disclosure;

FIG. 4 is a view for explaining an operation of a CBT-I treatment server according to an embodiment of the present disclosure; and

FIG. 5 illustrates a block diagram of a CBT-I treatment system according to another embodiment of the present disclosure.

### DETAILED DESCRIPTION OF THE DISCLOSURE

The embodiments will be described in detail herein with reference to the drawings.

However, it should be understood that the present disclosure is not limited to the embodiments according to the concept of the present disclosure, but includes changes, equivalents, or alternatives falling within the spirit and scope of the present disclosure.

In the following description of the present disclosure, a detailed description of known functions and configurations incorporated herein will be omitted when it may make the subject matter of the present disclosure unclear.

The terms used in the specification are defined in consideration of functions used in the present disclosure, and can be changed according to the intent or conventionally used methods of clients, operators, and users. Accordingly, definitions of the terms should be understood on the basis of the entire description of the present specification.

In description of the drawings, like reference numerals may be used for similar elements

The singular expressions in the present specification may encompass plural expressions unless clearly specified otherwise in context.

In this specification, expressions such as “A or B” and “at least one of A and/or B” may include all possible combinations of the items listed together.

Expressions such as “first” and “second” may be used to qualify the elements irrespective of order or importance, and are used to distinguish one element from another and do not limit the elements.

It will be understood that when an element (e.g., first) is referred to as being “connected to” or “coupled to” another element (e.g., second), it may be directly connected or coupled to the other element or an intervening element (e.g., third) may be present.

As used herein, “configured to” may be used interchangeably with, for example, “suitable for”, “ability to”, “changed to”, “made to”, “capable of”, or “designed to” in terms of hardware or software.

In some situations, the expression “device configured to” may mean that the device “may do˜” with other devices or components.

For example, in the sentence “processor configured to perform A, B, and C”, the processor may refer to a general-purpose processor (e.g., CPU or application processor) capable of performing corresponding operation by running a dedicated processor (e.g., embedded processor) for performing the corresponding operation, or one or more software programs stored in a memory device.

In addition, the expression “or” means “inclusive or” rather than “exclusive or”.

That is, unless otherwise mentioned or clearly inferred from context, the expression “x uses a or b” means any one of natural inclusive permutations.

Terms, such as “unit” or “module”, etc., should be understood as a unit that processes at least one function or operation and that may be embodied in a hardware manner, a software manner, or a combination of the hardware manner and the software manner.

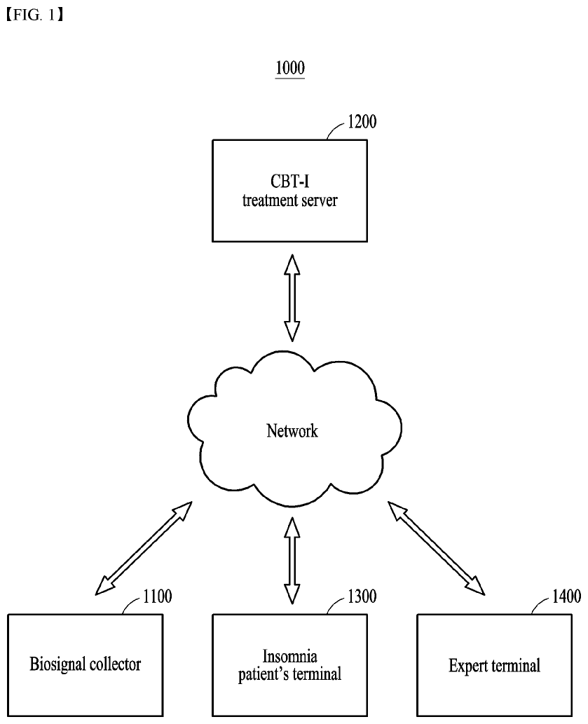

FIG. 1 is a view for explaining a CBT-I treatment system according to an embodiment of the present disclosure.

Referring to FIG. 1, a CBT-I treatment system 1000 according to an embodiment of the present disclosure includes a biometric collection device 1100, a CBT-I treatment server 1200, an insomnia patient's terminal 1300, and an expert terminal 1400. These components may exchange data with each other through a wired/wireless communication network.

The biometric collection device 1100 may generate biosignal data. Specifically, the biometric collection device 1100 may generate biosignal data by measuring a change in a biosignal during sleep of an insomnia patient using sensors attached to the patient's body. The biometric collection device 1100 may transmit the generated biosignal data to the CBT-I treatment server 1200.

In one embodiment, the biosignal data may include any a biosignal related to person's sleep such as electoencephalogram (EEG), electrocardiogram (ECG), electromyogram (EMG), electrooculogram (EOG), and photoplethysmogram (PPG). For this, the biometric collection device 1100 may include an electoencephalogram sensor, an electrocardiogram sensor, an electromyogram sensor, an electrooculogram sensor, a photoplethysmogram sensor, and the like.

The CBT-I treatment server 1200 generates a CBT-I prescription for insomnia treatment. Specifically, the CBT-I treatment server 1200 may receive biosignal data from the biometric collection device 1100 and may generate sleep evaluation data based on the biosignal data. The CBT-I treatment server 1200 may determine a CBT-I program for a sleep diary and insomnia treatment based on the sleep evaluation data. The CBT-I treatment server 1200 may generate a CBT-I prescription including the CBT-I program.

In one embodiment, the CBT-I treatment server 1200 may receive age information, gender information, smoking information, drinking level, exercise information, caffeine information, nap information, etc. of the insomnia patient from the insomnia patient's terminal 1300. Such information may be further included for determination of the CBT-I program.

In one embodiment, the CBT-I treatment server 1200 may transmit the CBT-I prescription to the expert terminal 1400 and may receive feedback from the expert terminal 1400. When correction information on the CBT-I prescription is included in the feedback, the CBT-I treatment server 1200 may generate a corrected CBT-I prescription by correcting the CBT-I program based on the correction information. The CBT-I treatment server 1200 may transmit the corrected CBT-I prescription to the insomnia patient's terminal 1300.

The insomnia patient's terminal 1300 provides content for insomnia treatment to an insomnia patient. Specifically, the insomnia patient's terminal 1300 may receive a CBT-I prescription from the CBT-I treatment server 1200, and may output a CBT-I program included in the received CBT-I prescription such that the insomnia patient can recognize using sight, hearing, etc.

In one embodiment, the insomnia patient's terminal 1300 may receive age information, gender information, smoking information, drinking level, exercise information, caffeine information, nap information, etc. from the insomnia patient and may transmit the received information to the CBT-I treatment server 1200.

In one embodiment, the insomnia patient's terminal 1300 may request information such as biosignal data, sleep evaluation data, sleep diary data, and a CBT-I prescription from the CBT-I treatment server 1200 when an information request command is input from the insomnia patient. When the insomnia patient's terminal 1300 receives biosignal data, sleep evaluation data, sleep diary data, a CBT-I prescription, etc. from the CBT-I treatment server 1200, the insomnia patient's terminal 1300 may output the biosignal data, sleep evaluation data, sleep diary data, CBT-I prescription, etc. so that the insomnia patient can recognize them.

In one embodiment, the insomnia patient's terminal 1300 may download and install an application for using the CBT-I treatment system 1000 from the CBT-I treatment server 1200 and other external servers. The insomnia patient's terminal 1300 may connect to the CBT-I treatment server 1200, etc. by executing the application.

The expert terminal 1400 is a terminal used by an expert, such as a doctor, with professional knowledge for insomnia treatment and may receive a CBT-I prescription from the CBT-I treatment server 1200. The expert terminal 1400 may output the received CBT-I prescription, etc. so that the expert can recognize it. The expert terminal 1400 may receive a confirmation command such as the need for correction or no correction by the expert for the CBT-I prescription. The expert terminal 1400 may transmit feedback including the confirmation command entered by the expert to the CBT-I treatment server 1200.

In addition, the insomnia patient's terminal 1300 and the expert terminal 1400 may be interconnected through a wired/wireless network to enable consultation and treatment of insomnia through video calls, voice calls, chatting, etc. between the insomnia patient and the expert.

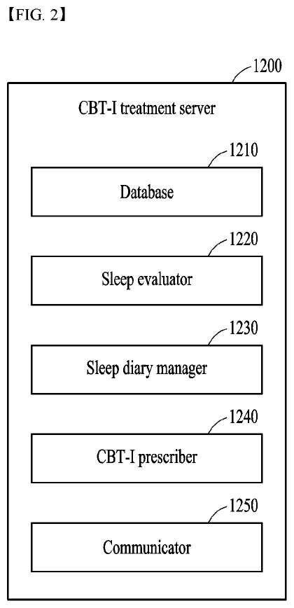

FIG. 2 illustrates a block diagram of a CBT-I treatment server according to an embodiment of the present disclosure.

Referring to FIG. 2, the CBT-I treatment server 1200 according to an embodiment of the present disclosure may include a database 2100, a sleep evaluator 2200, a sleep diary manager 2300, a CBT-I prescriber 2400 and a communicator 2500.

The database 2100 may store the CBT-I programs for insomnia treatment. Specifically, the database 2100 may store CBT-I programs, as shown in Table 1 below, for insomnia treatment dependent upon an insomnia patient's status, age, gender, etc.

TABLE 1

| CBT-1 | |
|---|---|
| program | Description |
| Sleep | Explain the basic structure (sleep steps 1 to 3, REM sleep) |
| Education | of sleep and explain the two processes (principle of sleep |
| | desire and circadian rhythm) of sleep. |
| Sleep | Education to follow for effective sleep. Ex) avoid excessive |
| Hygiene | alcohol-smoking-exercise before bedtime, avoid caffeine |
| | intake 6 hours before bedtime, limit naps, etc. |
| Stimulus | Re-learn associations with bed and sleep. Ex) Going to bed |
| Control | only when you are sleepy, getting out of bed if you cannot |
| | sleep, avoiding activities other than sleeping and sex in bed, |
| | etc. |
| Sleep | Limit time in bed to increase sleep efficiency over 85% |
| Restriction | (actual sleep time/time in bed). |
| Relaxation | Offer muscle relaxation, breathing therapy, or meditation |
| Techniques | therapy to reduce physical tension. |
| Cognitive | Correct any negative or erroneous thoughts about sleep. Ex) |
| Therapy | Belief that 8 hours of sleep is necessary for ideal sleep. |
| Light | Control circadian rhythm |
| Therapy | |

In one embodiment, the CBT-I programs may be content such as video, image, and voice.

In one embodiment, the database 2100 may store individual data of an insomnia patient. Specifically, the database 2100 may include information such as age information, gender information, smoking information, drinking level, exercise information, caffeine information, and nap information of each insomnia patient, biosignal data, sleep evaluation data, sleep diary data, a CBT-I prescription, expert feedback, and the like.

The sleep evaluator 2200 may generate sleep evaluation data for an insomnia patient. Specifically, the sleep evaluator 2200 may generate sleep evaluation data based on biosignal data including a change in a biosignal during sleep of an insomnia patient.

In one embodiment, the sleep evaluator 2200 may generate sleep evaluation data from biosignal data using Time In Bed (TIB), Total Sleep Time (TST), Wake time After Sleep Onset (WASO), Sleep On Latency (SOL), Sleep Efficiency (SE), Number of Awakenings (NWAK), etc. as evaluation indexes. Here, SOL is an index considered when diagnosing erectile dysfunction when it is more than 30 minutes based on 30 minutes from in-bed time to entering a sleep state, and for SE, a total sleep time/total in-bed time of 85% may be an indicator of insomnia treatment criteria. That is, when SE is 85% or less, cognitive behavioral therapy may be performed so that it becomes 85% or more.

In one embodiment, the sleep evaluation data may include data such as sleep time, time taken to enter sleep, and degree of deep sleep.

In one embodiment, the sleep evaluator may generate sleep evaluation data using the algorithm for analyzing sleep stages using heart rates and wrist movement described in the paper “Assessing sleep architecture and continuity measures through the analysis of heart rate and wrist movement recordings in healthy subjects: comparison with results based on polysomnography (Sleep Med. 2016 May; 21:47-56.)” published in 2200.

In one embodiment, the sleep evaluator 2200 may generate sleep evaluation data using an algorithm for analyzing REM sleep stages using heart rate variability (HRV) derived from heart rates described in the published paper “REM sleep estimation based on autonomic dynamics using R-R intervals (Published 17 Mar. 2017, © 2017 Institute of Physics and Engineering in Medicine).”

In one embodiment, the sleep evaluator 2200 may generate sleep evaluation data by using an algorithm for classifying a sleep state and wakefulness state from the movement of a patient described in the published paper “Activity-Based Sleep-Wake Identification: An Empirical Test of Methodological Issues (Sleep. 1994 April; 17(3):201-7.).”

The sleep diary manager 2300 manages a sleep diary of an insomnia patient. Specifically, the sleep diary manager 2300 may generate an insomnia patient's sleep diary data based on sleep evaluation data for the insomnia patient. The sleep diary manager 2300 may store the sleep diary data in the database 2100.

The CBT-I prescriber 2400 creates a CBT-I prescription for insomnia treatment. Specifically, the CBT-I prescriber 2400 may determine a CBT-I program for treating patient's insomnia among a plurality of CBT-I programs stored in the database 2100 based on the sleep evaluation data for the insomnia patient. The CBT-I prescriber 2400 may generate a CBT-I prescription including the determined CBT-I program.

In one embodiment, when the CBT-I prescriber 2400 receives a feedback including correction information on the CBT-I program included in the CBT-I prescription from the expert terminal 1400, based on the correction information, the CBT-I prescriber 2400 may correct the CBT-I program based on the correction information. The CBT-I prescriber 2400 may generate a corrected CBT-I prescription containing the corrected CBT-I program.

In one embodiment, the CBT-I prescriber 2400 may generate a CBT-I prescription including a new CBT-I program based on the insomnia patient's sleep evaluation data after the CBT-I prescription previously provided in the insomnia patient's terminal 1300.

The communicator 2500 performs communication between the CBT-I treatment server 1200 and an external device. Specifically, the communicator 2500 may perform data communication with external devices such as the biometric collection device 1100, the insomnia patient's terminal 1300, and the expert terminal 1400.

In one embodiment, the communicator 2500 may receive biosignal data including a change in a biosignal during sleep of the insomnia patient from the biometric collection device 1100.

In one embodiment, the communicator 2500 may transmit a CBT-I prescription, sleep diary data, sleep evaluation data, etc. to the insomnia patient's terminal 1300. The communicator 2500 may receive information such as age information, gender information, smoking information, drinking level, exercise information, caffeine information, and nap information of the insomnia patient from the insomnia patient's terminal 1300.

In one embodiment, the communicator 2500 may transmit the CBT-I prescription, the sleep diary data, the sleep evaluation data, etc. to the expert terminal 1400. The communicator 2500 may receive feedback, which includes information on whether to correct the CBT-I prescription, from the expert terminal 1400.

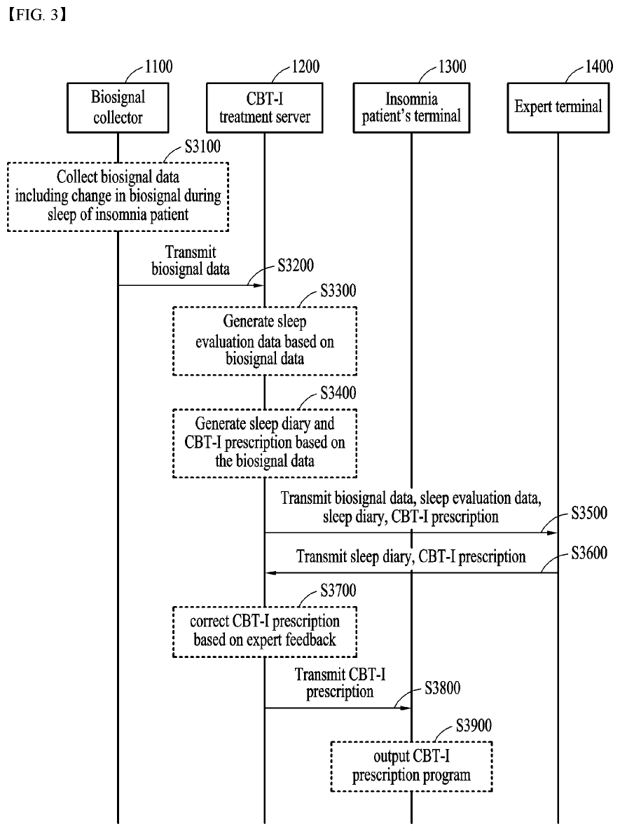

FIG. 3 is a view for explaining an operation of a CBT-I treatment system according to an embodiment of the present disclosure.

In S3100, biosignal data is collected. Specifically, the biometric collection device 1100 may collect biosignal data including a change in a biosignal during sleep of an insomnia patient by using sensors attached to the insomnia patient's body.

In S3200, the biometric collection device 1100 may transmit biosignal data to the CBT-I treatment server 1200. The CBT-I treatment server 1200 may receive the biosignal data from the biometric collection device 1100.

In S3300, sleep evaluation data is generated. Specifically, the CBT-I treatment server 1200 may generate sleep evaluation data for the insomnia patient based on the biosignal data received from the biometric collection device 1100.

In one embodiment, the CBT-I treatment server 1200 may generate sleep evaluation data from the biosignal data using Time In Bed (TIB), Total Sleep Time (TST), Wake time After Sleep Onset (WASO), Sleep On Latency (SOL), Sleep Efficiency (SE), Number of Awakenings (NWAK), etc. as evaluation indexes.

In S3400, a CBT-I prescription is generated. Specifically, the CBT-I treatment server 1200 may determine a CBT-I program for treating the insomnia patient based on the sleep evaluation data. The CBT-I treatment server 1200 may generate a CBT-I prescription including the determined CBT-I program.

In addition, the CBT-I treatment server 1200 may generate sleep diary data including sleep diary information of the insomnia patient based on the sleep evaluation data.

In S3500, the CBT-I treatment server 1200 may transmit biosignal data, sleep evaluation data, sleep diary data, a CBT-I prescription, etc. to the expert terminal 1400.

In S3600, the expert terminal 1400 may out the biosignal data, sleep evaluation data, sleep diary data, CBT-I prescription, etc. received from the CBT-I treatment server 1200 such that an expert can recognize it. A confirmation command including information on whether to correct the biosignal data, the sleep evaluation data, the sleep diary data, the CBT-I prescription, etc. may be input in the expert terminal 1400 by the expert. The expert terminal 1400 may transmit feedback including the inputted confirmation command, etc. to the CBT-I treatment server 1200.

In S3700, the CBT-I prescription is corrected. Specifically, when the feedback received from the expert terminal 1400 includes correction information on the CBT-I prescription, the CBT-I treatment server 1200 may correct the CBT-I program based on the correction information and may generate a corrected CBT-I prescription including the corrected CBT-I program.

In addition, the CBT-I treatment server 1200 may not correct the CBT-I prescription when the feedback received from the expert terminal 1400 does not include correction information on the CBT-I prescription.

In S3800, the CBT-I treatment server 1200 may transmit the corrected CBT-I prescription to the insomnia patient's terminal 1300. The insomnia patient's terminal 1300 may receive the corrected CBT-I prescription from the CBT-I treatment server 1200.

In addition, the CBT-I treatment server 1200 may transmit an uncorrected CBT-I prescription to the insomnia patient's terminal 1300 when the feedback received from the expert terminal 1400 does not contain correction information on the CBT-I prescription. may transmit. The insomnia patient's terminal 1300 may receive the uncorrected CBT-I prescription from the CBT-I treatment server 1200.

In S3900, the insomnia patient's terminal 1300 may output the corrected CBT-I program (or non-corrected CBT-I program) included in the CBT-I prescription received from the CBT-I treatment server 1200 such that the insomnia patient can recognize using sight, hearing, etc.

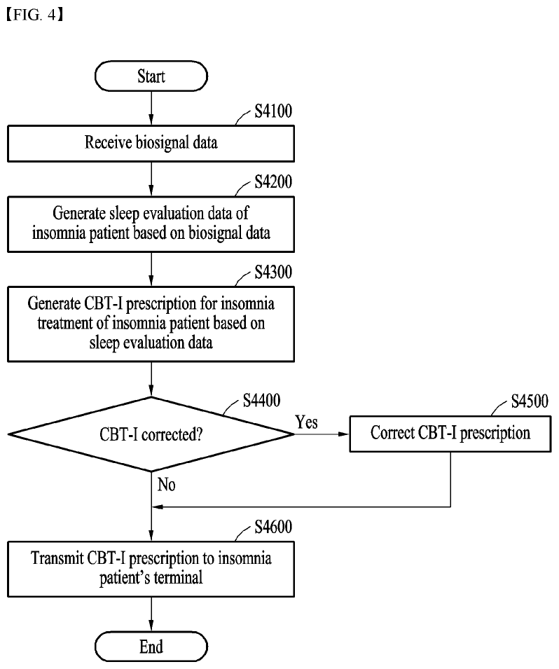

FIG. 4 is a view for explaining an operation of a CBT-I treatment server according to an embodiment of the present disclosure.

In S4100, biosignal data is received. Specifically, the CBT-I treatment server 1200 may receive biosignal data including a change in a biosignal during sleep of an insomnia patient from the biometric collection device 1100.

In S4200, sleep evaluation data is generated. Specifically, the CBT-I treatment server 1200 may generate sleep evaluation data for the insomnia patient based on the biosignal data.

In one embodiment, the CBT-I treatment server 1200 may generate sleep evaluation data from the biosignal data using Time In Bed (TIB), Total Sleep Time (TST), Wake time After Sleep Onset (WASO), Sleep On Latency (SOL), Sleep Efficiency (SE), Number of Awakenings (NWAK), etc. as evaluation indexes.

In S4300, a CBT-I prescription is generated. Specifically, the CBT-I treatment server 1200 may determine a CBT-I program for treating the insomnia patient based on the sleep evaluation data for the insomnia patient. The CBT-I treatment server 1200 may generate a CBT-I prescription including the determined CBT-I program.

In S4400, it is determined whether to correct the CBT-I prescription. Specifically, the CBT-I treatment server 1200 may transmit the CBT-I prescription, etc. to the expert terminal 1400. The CBT-I treatment server 1200 may receive expert's feedback including information on whether or not to correct the CBT-I prescription from the expert terminal 1400. The CBT-I treatment server 1200 may determine that correction for the CBT-I prescription is necessary if correction information is included in the feedback received from the expert terminal 1400.

In addition, the CBT-I treatment server 1200 may determine that there is no need to correct the CBT-I prescription if correction information is not included in the feedback received from the expert terminal 1400.

In S4500, the CBT-I prescription is corrected. Specifically, the CBT-I treatment server 1200 may correct the CBT-I program based on the correction information included in the feedback received from the expert terminal 1400 when it is determined that correction of the CBT-I prescription is necessary, and may generate a corrected CBT-I prescription including the corrected CBT-I program.

In S4600, the CBT-I prescription is transmitted. Specifically, the CBT-I treatment server 1200 may transmit the CBT-I prescription to the expert terminal 1400.

In addition, when the CBT-I prescription is corrected, the CBT-I treatment server 1200 may transmit the corrected CBT-I prescription to the expert terminal 1400.

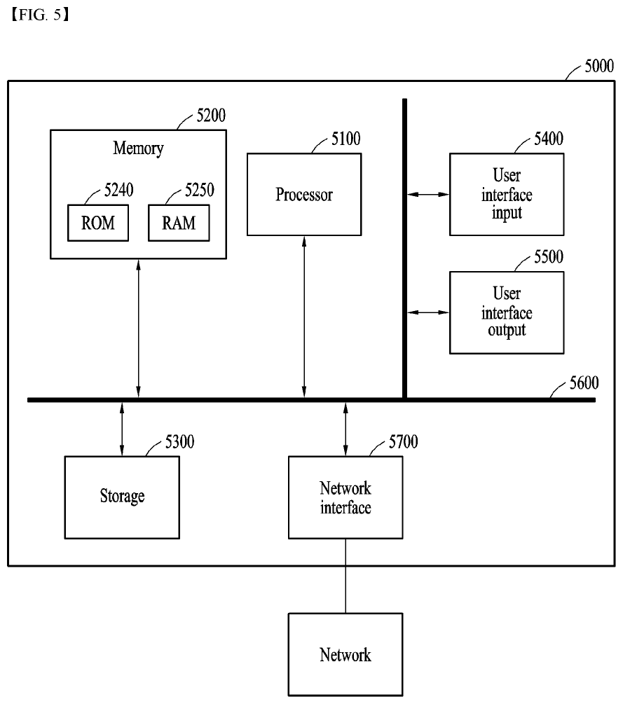

FIG. 5 illustrates a block diagram of a CBT-I treatment system according to another embodiment of the present disclosure.

As shown in FIG. 5, the biometric collection device 1100, the CBT-I treatment server 1200, the insomnia patient's terminal 1300, and the expert terminal 1400 may include at least one element of a processor 5100, a memory 5200, a storage 5300, a user interface input 5400 and a user interface output 5500, and these elements may communicate with each other through a bus 5600. In addition, the biometric collection device 1100, the CBT-I treatment server 1200, the insomnia patient's terminal 1300, and the expert terminal 1400 may also include a network interface 5700 for accessing a network. The processor 5100 may be a CPU or semiconductor element that executes processing instructions stored in the memory 5200 and/or the storage 5300. The memory 5200 and the storage 5300 may include various types of volatile/nonvolatile memory media. For example, the memory may include ROM 5240 and RAM 5250.

In accordance with an aspect of the present disclosure, insomnia treatment that reflects the individual characteristics of insomnia patients can be provided.

In addition, in accordance with another aspect of the present disclosure, insomnia can be treated without requiring an insomnia patient to visit a hospital or face a specialist such as a doctor.

The methods according to the embodiments of the present disclosure may be implemented in the form of a program command that can be executed through various computer means and recorded in a computer-readable medium. The computer-readable medium can store program commands, data files, data structures or combinations thereof. The program commands recorded in the medium may be specially designed and configured for the present disclosure or be known to those skilled in the field of computer software. Examples of a computer-readable recording medium include magnetic media such as hard disks, floppy disks and magnetic tapes, optical media such as CD-ROMs and DVDs, magneto-optical media such as floptical disks, or hardware devices such as ROMs, RAMs and flash memories, which are specially configured to store and execute program commands. Examples of the program commands include machine language code created by a compiler and high-level language code executable by a computer using an interpreter and the like. The hardware devices described above may be configured to operate as one or more software modules to perform the operations of the embodiments, and vice versa.

The software may include computer programs, code, instructions, or a combination of one or more of the foregoing, configure the processing apparatus to operate as desired, or command the processing apparatus, either independently or collectively. In order to be interpreted by a processing device or to provide instructions or data to a processing device, the software and/or data may be embodied permanently or temporarily in any type of a machine, a component, a physical device, a virtual device, a computer storage medium or device, or a transmission signal wave. The software may be distributed over a networked computer system and stored or executed in a distributed manner. The software and data may be stored in one or more computer-readable recording media.

Although the present disclosure has been described with reference to limited embodiments and drawings, it should be understood by those skilled in the art that various changes and modifications may be made therein. For example, the described techniques may be performed in a different order than the described methods, and/or components of the described systems, structures, devices, circuits, etc., may be combined in a manner that is different from the described method, or appropriate results may be achieved even if replaced by other components or equivalents.

Therefore, other embodiments, other examples, and equivalents to the claims are within the scope of the following claims.

What is claimed is:

1. A CBT-I treatment server, comprising:
a database configured to store a plurality of CBT-I programs for insomnia treatment;
a sleep evaluator configured to generate sleep evaluation data of an insomnia patient based on biosignal data comprising a change in a biosignal during sleep of the insomnia patient;
a CBT-I prescriber configured to determine a CBT-I program for insomnia treatment of the insomnia patient from among the plural CBT-I programs based on the sleep evaluation data and to generate a CBT-I prescription comprising the determined CBT-I program; and
a communicator configured to receive a biosignal from a biosignal collection device for generating biosignal data by sensing a change in a biosignal during sleep of the insomnia patient and to transmit the CBT-I prescription to an insomnia patient's terminal.

2. The CBT-I treatment server according to claim 1, wherein the biosignal data comprises at least one of electoencephalogram (EEG), electrocardiogram (ECG), electromyogram (EMG), electrooculogram (EOG) and photoplethysmogram (PPG).

3. The CBT-I treatment server according to claim 1, wherein the sleep evaluator generates the sleep evaluation data according to at least one indicator of Time In Bed (TIB), Total Sleep Time (TST), Wake time After Sleep Onset (WASO), Sleep On Latency (SOL), Sleep Efficiency (SE) and Number of Awakenings (NWAK).

4. The CBT-I treatment server according to claim 1, further comprising a sleep diary manager configured to generate a sleep diary of the insomnia patient based on the sleep evaluation data,
wherein the communicator transmits the sleep diary to the insomnia patient's terminal.

5. The CBT-I treatment server according to claim 1, wherein the communicator further receives at least one of age information, gender information, smoking information, drinking level, exercise information, caffeine information, and nap information of the insomnia patient from the insomnia patient's terminal, and
the CBT-I prescriber further comprises at least one of age information, gender information, smoking information, drinking level, exercise information, caffeine information, and nap information of the insomnia patient and determines a CBT-I program for insomnia treatment of the patient.

6. The CBT-I treatment server according to claim 1, wherein the communicator transmits the CBT-I prescription to an expert terminal and receives feedback, inputted by an insomnia treatment expert, for the CBT-I prescription from the expert terminal.

7. The CBT-I treatment server according to claim 6, wherein the CBT-I prescriber, when the feedback comprises correction information on the CBT-I prescription, corrects the determined CBT-I program based on the correction information, and generates a corrected CBT-I prescription comprising the corrected CBT-I program, and
the communicator transmits the corrected CBT-I prescription to the insomnia patient's terminal.

8. The CBT-I treatment server according to claim 6, wherein, when the feedback does not comprise correction information on the CBT-I prescription, the communicator transmits the CBT-I prescription comprising the determined CBT-I program to the insomnia patient's terminal.

9. A method of operating a CBT-I treatment server comprising a database for storing a plurality of CBT-I programs for insomnia treatment, the method comprising:
receiving a biosignal from a biosignal collection device for collecting biosignal data comprising a change in a biosignal during sleep of an insomnia patient;
generating sleep evaluation data of the insomnia patient based on the biosignal data comprising the change in the biosignal during sleep of the insomnia patient;
determining a CBT-I program for insomnia treatment of the insomnia patient from among the plural CBT-I programs based on the sleep evaluation data and generating a CBT-I prescription comprising the determined CBT-I program; and
transmitting the CBT-I prescription to an insomnia patient's terminal.

10. The method according to claim 9, wherein the biosignal data comprises at least one of electoencephalogram (EEG), electrocardiogram (ECG), electromyogram (EMG), electrooculogram (EOG) and photoplethysmogram (PPG).

11. The method according to claim 9, wherein, in the generating of the sleep evaluation data, the sleep evaluation data is generated according to at least one indicator of Time In Bed (TIB), Total Sleep Time (TST), Wake time After Sleep Onset (WASO), Sleep On Latency (SOL), Sleep Efficiency (SE) and Number of Awakenings (NWAK).

12. The method according to claim 9, further comprising generating a sleep diary of the insomnia patient based on the sleep evaluation data,
wherein the transmitting of the CBT-I prescription further comprises transmitting the sleep diary to the insomnia patient's terminal.

13. The method according to claim 9, further comprising receiving at least one of age information, gender information, smoking information, drinking level, exercise information, caffeine information, and nap information of the insomnia patient from the insomnia patient's terminal,
wherein, in the generating of the CBT-I prescription, at least one of age information, gender information, smoking information, drinking level, exercise information, caffeine information, and nap information of the insomnia patient is further comprised and a CBT-I program for insomnia treatment of the patient is determined.

14. The method according to claim 9, further comprising transmitting the CBT-I prescription to an expert terminal and receiving feedback, inputted by an insomnia treatment expert, for the CBT-I prescription from the expert terminal.

15. The method according to claim 14, further comprising, when the feedback comprises correction information on the CBT-I prescription, correcting the determined CBT-I program based on the correction information, and generating a corrected CBT-I prescription comprising the corrected CBT-I program,
wherein, in the transmitting of the CBT-I prescription, the corrected CBT-I prescription is transmitted to the insomnia patient's terminal.

16. The method according to claim 14, wherein, in the transmitting of the CBT-I prescription, the CBT-I prescription comprising the determined CBT-I program is transmitted to the insomnia patient's terminal when the feedback does not comprise correction information on the CBT-I prescription.

17. A CBT-I treatment system, comprising:
a biosignal collector configured to collect biosignal data comprising a change in a biosignal during sleep of an insomnia patient and to transmit the collected biosignal data;
a CBT-I treatment server configured to receive the biosignal data from the biosignal collector to generate sleep evaluation data of the insomnia patient based on the biosignal data, to determine a CBT-I program for insomnia treatment of the patient from among the plural CBT-I programs based on the sleep evaluation data, and to generate a CBT-I prescription comprising the determined CBT-I program; and
an insomnia patient's terminal configured to receive the CBT-I prescription from the CBT-I treatment server and to output the CBT-I program comprised in the CBT-I prescription.
